(19) United States
(12) Patent Application Publication
(10) Pub. No.: US 2015/0150902 A1
(43) Pub. Date: Jun. 4, 2015

(54) COMPOSITION AND METHOD FOR PROMOTING JOINT AND BONE HEALTH

(71) Applicant: NEXGEN PHARMA, INC., Irvine, CA (US)

(72) Inventor: Brian A. CROOK, Colorado Springs, CA (US)

(21) Appl. No.: 14/611,130

(22) Filed: Jan. 30, 2015

Related U.S. Application Data
(63) Continuation of application No. 13/476,880, filed on May 21, 2012.

(51) Int. Cl. A61K 33/00; A61K 31/737; A61K 31/10; A61K 31/7008

(52) U.S. Cl. CPC A61K 33/00; A61K 31/7008; A61K 31/737; A61K 31/10

(57) ABSTRACT

The present invention provides compositions comprising silicon and at least one other joint health ingredient such as glucosamine, chondroitin, hyaluronic acid, collage, etc. as well as methods for making and using the same.

### FIELD OF THE INVENTION

The present invention relates generally to a composition comprising silicon and glucosamine, methods for making and using the same. In some embodiments, compositions of the invention are provided as dietary supplements comprising the same.

### BACKGROUND OF THE INVENTION

Deterioration of joints and bone health and strength is one of the major causes of debilitation in both human and animals. Joint deterioration and poor bone health in some animals, e.g., equines, is particularly detrimental. There are numerous commercial products that include glucosamine, hyaluronic acid, collagen, chondroitin, methylsulfonylmethane, avocado/soybean unsaponifiables, sodium hyaluronate, selenium, zinc, or a combination thereof for supporting joint health. However, by only supporting joint health, and not addressing bone health and strength, these products often leave the human or animal susceptible to injury and poor athletic performance.

While a variety of compositions are known to support joint health, these are not always effective in improving joint health while improving bone health and strength resulting in improved athletic performance. Accordingly, there is a continuing need for a composition for improving joint health, bone health and strength and athletic performance.

### SUMMARY OF THE INVENTION

Various aspects of the invention comprise compositions that are useful for joint health, mineral metabolism, bone health, bone strength and/or improved athletic performance in a mammal and methods for using the same.

In one particular aspect, the composition of the invention comprises a therapeutically effective amount of silicon and glucosamine. Typically, the composition of the invention comprises about 0.1 mg to about 1 gram of silicon. In another embodiments, the composition of the invention comprises from about 10 mg to about 100 grams of glucosamine.

Still in other embodiments, the composition of the invention is in a single unit form, often a single dosage unit form, i.e., once a daily dosage form. In other embodiments, the composition of the invention is orally administrable.

Yet in other embodiments, the composition of the invention further comprises chondroitin, methylsulfonylmethane, hyaluronic acid, or a combination thereof. When present, a typical amount of chondroitin in the composition ranges from about 5 mg to about 100 grams of chondroitin. When present, a typical amount of methylsulfonylmethane in the composition ranges from about 5 mg to about 100 grams.

In other embodiments, the ratio of glucosamine to silicon ranges from about 600:1 to about 7200:1. While a variety of silicon sources are available that can be used in the present invention, in one particular embodiment, silicon is selected from the group consisting of potassium silicate, sodium silicate, calcium silicate, sodium aluminum silicate, sodium zeolite A, orthosilicic acid, silicic acid, and a combination thereof. However, it should be appreciated that the scope of the invention is not limited to these silicon sources. Any suitable silicon source can be used in compositions of the invention.

Other aspects of the invention provide compositions consisting essentially of a therapeutically effective amount of silicon, glucosamine; optionally chondroitin, optionally methylsulfonylmethane, optionally hyaluronic acid; optionally collagen; and optionally a pharmaceutically acceptable excipient. In some embodiments, such compositions consist essentially of a therapeutically effective amount of silicon, glucosamine; optionally chondroitin, optionally methylsulfonylmethane, and optionally a pharmaceutically acceptable excipient. Still in other embodiments, such compositions consist essentially of a therapeutically effective amount of silicon, glucosamine; and optionally a pharmaceutically acceptable excipient. In some embodiments, the amount and/or the ratio of individual components of the compositions are those disclosed herein. However, it should be appreciated that the scope of the invention is not limited to those specific amount and/or the ratio of individual components disclosed herein.

Still other aspects of the invention provide methods for increasing joint health, mineral metabolism, bone health, bone strength or performance in a mammal by administering to the mammal a therapeutically effective amount of a composition disclosed herein.

In some embodiments the mammal is selected from the group consisting of human, non-human primate, equine, bovine, canine, feline, caprine, ovine and rodent.

Yet in other embodiments, the composition is administered to the mammal once a day. The composition can be administered orally or parenterally. In some embodiments, the composition comprises from about 0.1 mg to about 10 grams of silicon. Yet in other embodiments, the composition comprises from about 10 mg to about 100 grams of glucosamine.

Other aspects of the invention provide methods for improving a joint performance, mineral metabolism, bone health, bone strength and/or athletic performance in a mammal comprising administering to the mammal a therapeutically effective amount of a composition disclosed herein.

Still other aspects of the invention provide methods for treating a clinical condition associated joint pain, joint stiffness, joint inflammation, joint deterioration, or a combination thereof, said method comprising administering to a mammal in need of such a treatment a composition disclosed herein. In one particular embodiment, the composition comprises silicon and glucosamine.

In other aspects of the invention, methods of supporting joint health or treatment of joint deterioration from osteoarthritis, degenerative joint disease, joint defect, rheumatoid arthritis or a combination thereof in a mammal is provided. Such methods comprise administering to the mammal in need of such a support a composition disclosed herein.

### DETAILED DESCRIPTION OF THE INVENTION

It is generally believed that silicon forms about 28% of the total earth crust and is, after oxygen, the most common atom on earth. In nature silicon doesn't occur as a free atom. Silicon can be found, e.g., in the form of sand, pebble, rock, quartz, granite and clay. Silicon compounds are generally found as silicates (SiO₂ and derivates), silicides (silicon compounds with carbon), silanoles (compounds of silicon with hydrogen), silicons (polymers of silicon with oxygen), etc. Until the 1970's silicon was not considered to be essential for the metabolism of humans because only a relatively low concentrations of silicon were found in human tissue.

Silicon (i.e., silica) is an essential mineral and enhancer of other minerals such as calcium for bones, glucosamine for joints and antioxidants for healthier arteries and cardiovascular function. Silicon is a trace element for the formation of healthy connective tissue, bones, skin, hair and nails. Without being bound by any theory, it is believed that silicon improves the mineralization process in bones and has beneficial effects on bone quality. Silica is also believed to be important for collagen formation, healthy arteries and the regulation of calcium deposition in bone. The absorption is important to silicon's effectiveness, because majority of silica in food sources, and colloidal gel (silica) products are poorly absorbed because of their insoluble, polymerized forms. For effective absorption, silica diet must first be converted into organic silicon (e.g., monomethylsilanetriol). This form of silica has an excellent bioavailability and is available in some commercial products, such as Orgone LivingSilica.

While studies have shown that silicon (Si) supplementation is beneficial for mineral metabolism and bone health, to date no one has recognized the benefit of silicon, in combination with other nutrients, in the improvement in joint health, bone health and strength, and athletic performance.

Surprisingly and unexpectedly, the present inventors have discovered that silicon, in combination with other nutrients, significantly improves or aids in joint health, bone health and strength, and athletic performance in animals. As used herein, the term “animals” includes mammals such as equines, primates (including human), cattle, canines, felines, as well as other domesticated mammals and rodents.

Some aspects of the invention provide a composition for joint healing, bone health and strength, and athletic performance that comprises silicon and at least one other ingredient that is beneficial for joints and bone health. Typically, the other ingredient comprises chondroitin, hyaluronic acid, collagen, glucosamine, methylsulfonylmethane, avocado/soybean unsaponifiables, sodium hyaluronate, selenium, and zinc, or a mixture thereof. In some embodiments, the other ingredient comprises chondroitin, glucosamine, or a mixture thereof. In one particular embodiment, the other ingredient is glucosamine.

Compositions of the invention provide a convenient form for administering silicon and other ingredient(s) simultaneously that are beneficial for joint health, bone health and strength, and athletic performance by providing the mixture as a single dosage form.

Accordingly, some embodiments of the invention provide a single dosage composition comprising silicon and at least one other ingredient that is beneficial for joint health, bone health and strength, and athletic performance. Surprisingly and unexpectedly, the present inventors have discovered that co-administration of silicone with other ingredient(s) that are beneficial for joint results in a synergistic effect of providing a higher efficacy and/or efficiency for joint health, bone health and strength, and athletic performance than administering these ingredients separately at different times.

The source of silicon can be any known material including various alkaline silicates, alkaline earth silicates, and other metallic silicates. The source of silicon can also be organic compounds. In some embodiments, the source of silicon is alkaline silicate, alkaline earth silicate or a transition metal silicate. Exemplary alkaline silicate, alkaline earth silicate, and transition metal silicates include, but are not limited to, potassium silicate, sodium silicate, calcium silicate, lithium silicate, magnesium silicate, cesium silicate, zeolites, as well as others.

While the recommended daily allowance of silicon has not yet been established, it has been found by the present inventors that for equine the amount of silicon suitable for joint health benefit is at least 1 mg/100 Kg-day, typically at least 4 mg/100 Kg-day, and often at least 8 mg/100Kg-day. Alternatively, the amount of silicon for joint health, bone health and strength, and athletic performance benefit ranges from about 1 mg/100 Kg-day to about 12 mg/100 Kg-day, typically from about 2 mg/100 Kg-day to about 10 mg/100 Kg-day, and often from about 4 mg/100 Kg-day to about 8 mg/100 Kg-day. It should be appreciated, however, that the scope of the invention is not limited to these particular amount or ranges. The actual amount of silicon for joint health, bone health and strength, and athletic performance benefit can vary significantly depending on the animal and on animal's age, sex, weight, the nature of silicon material administered, etc.

In some embodiments, in addition to silicon, compositions of the invention include glucosamine. For equines, the ratio of glucosamine to silicon in a single dosage form is at least about 600:1, typically at least 1800:1, and often at least 3600:1. Alternatively, the ratio of glucosamine to silicon ranges from about 600:1 to about 7200:1, typically from about 1800:1 to about 5400:1, and often from about 1800:1 to about 3600:1. It should be appreciated that the scope of the invention is not limited to these particular ratio between glucosamine and silicon. The ratio of glucosamine to silicon can vary significantly depending on a variety of factors such as the animal and on animal's age, sex, weight, the form of glucosamine and silicon material administered, etc. Glucosamine can be obtained from any of the sources known to one skilled in the art. Still alternatively, the amount of glucosamine in compositions of the invention for equines can be from about 5 grams to about 100 grams, typically from about 20 grams to about 75 grams, and often from about 25 to about 60 grams. In other embodiments, the amount of glucosamine in compositions of the invention is at least about 250 mg, typically at least about 600 mg, and often at least about 800 mg.

Yet in other embodiments, compositions of the invention can also optionally include chondroitin. It should be appreciated that when chondroitin is present in the composition of the invention, glucosamine can also be included, but it is not necessary. For equines, the ratio of chondroitin to silicon is at least about 100:1, typically at least 300:1, and often at least 600:1. Alternatively, the ratio of chondroitin to silicon ranges from about 50:1 to about 900:1, typically from about 150:1 to about 500:1, and often from about 250:1 to about 350:1. It should be appreciated that the scope of the invention is not limited to these particular ratio between chondroitin and silicon. The ratio of chondroitin to silicon can vary significantly depending on a variety of factors such as the animal and on animal's age, sex, weight, the form of chondroitin and/or silicon material administered, etc. Chondroitin can be obtained from any of the sources known to one skilled in the art. In some embodiments, the amount of chondroitin in compositions of the invention for equines is from about 250 mg to about 15000 mg, typically from about 2500 mg to about 15000 mg, and often from about 5000 mg to about 10000 mg. Yet in other embodiments, the amount of chondroitin in compositions of the invention is at least about 50 mg, typically at least about 600 mg, and often at least about 1200 mg.

When both chondroitin and glucosamine are present in the compositions of the invention, for equines, the ratio of glucosamine to chondroitin is at least about 1:1, typically at least 3:1, and often at least 6:1. Alternatively, the ratio of chondroitin to glucosamine ranges from about 0.5:1 to about 8:1, typically from about 3:1 to about 7:1, and often from about 5:1 to about 6:1. It should be appreciated that the scope of the invention is not limited to these particular ratio between glucosamine and chondroitin. The ratio of glucosamine to chondroitin can vary significantly depending on a variety of factors such as the type of animal, animal's age, sex, weight, the form of glucosamine and/or chondroitin material administered, etc.

Compositions of the invention can also include other ingredients that are beneficial to joint health. Such ingredients include hyaluronic acid, collagen, methylsulfonylmethane (i.e., MSM), avocado/soybean unsaponifiables, sodium hyaluronate, selenium, and zinc, as well as other joint health ingredients that are known to one skilled in the art. As used herein, the term “joint health ingredient” refers to any ingredient that is beneficial to or supporting health of joints. The amount of each joint health ingredients can vary depending on a variety of factors including, but not limited to, the type of animal, age of the animal, sex of the animal, the weight of the animal, the form of joint health ingredient used to administer the composition, etc.

Still in other embodiments, compositions of the invention can also include other nutritional supplements such as vitamins (e.g., vitamin C, vitamin D, Vitamin B's, etc.), as well as minerals, etc. Exemplary minerals that can be present in compositions of the invention include, but are not limited to, calcium, zinc, iron, copper and magnesium, and their pharmaceutically accepted salts.

Compositions of the invention can also include other desirable additives such as, but not limited to, starches, sugars, fats, antioxidants, amino acids, proteins, derivatives thereof or combinations thereof Antioxidants generally improve the stability of the final composition. Inclusion of additives that assist in formulating the final composition are also desirable.

Compositions of the invention are typically formulated as a single dosage form that can be administered orally once a day to achieve a beneficial effect on joints. However, it should be appreciated that different formulations (e.g., parenteral administration form, two or more times per day administration dosage) are also within the scope of the invention.

Some aspects of the invention provide orally administrable single dosage compositions including silicon and at least one other joint health ingredient such as chondroitin, glucosamine, hyaluronic acid, collagen, etc. Generally, animals such as mammals will benefit by consuming silicon and other joint health ingredient included in the present compositions. In some embodiments, compositions of the invention are directed to equines.

In general, the compounds of the invention are administered in a therapeutically effective amount by any of the accepted modes of administration for agents that serve similar utilities. One of ordinary skill in the art of treating clinical conditions associated with joint is typically able, without undue experimentation and in reliance upon personal knowledge and the disclosure of this application, to ascertain a therapeutically effective amount of the compounds of the invention.

Typically, compositions of the invention are administered as nutraceutical or pharmaceutical formulations including those suitable for oral or parenteral administration. Typical manner of administration is generally oral using a convenient daily dosage regimen.

Compositions of the invention, together with one or more conventional adjuvants, carriers, or diluents, can be placed into the form of nutraceutical/pharmaceutical compositions and unit dosages. The nutraceutical/pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. Compositions of the invention can be employed as solids, such as tablets or filled capsules, semisolids, powders, sustained release formulations, or liquids such as solutions, suspensions, emulsions, elixirs, or filled capsules for oral use; or in the form of sterile injectable solutions for parenteral use.

Compositions of the invention can be formulated in a wide variety of oral administration dosage forms. Compositions and dosage forms can comprise silicon and other joint health ingredient(s) as the active components. The nutraceutically/pharmaceutically acceptable carriers can be either solid or liquid. Solid form preparations include powders, tablets, pills, capsules, cachets, soft chews, boluses, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets typically contain from about one (1) to about seventy (70) percent of the active composition (i.e., silicon and other joint health ingredient(s)). Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active composition with encapsulating material as carrier, providing a capsule in which the active composition, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, semi-solid forms, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active composition (i.e., component) in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

Compositions of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the composition can be in powder form, obtained by aseptic isolation of sterile solid or by lyophilization from solution for constitution before use with a suitable vehicle, e.g., sterile, pyrogen-free water.

When desired, formulations can be prepared with enteric coatings adapted for sustained or controlled release administration of the active ingredients (i.e., compositions of the invention).

Compositions of the invention are typically prepared in unit dosage forms, often in a single unit dosage form. As used herein, the term “single unit dosage form” refers to a unit dosage form that is designed to be administered once a day. In unit dosage forms, the preparation is often subdivided into unit doses containing appropriate quantities of the composition of the invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable carriers and their formulations are described in Remington: The Science and Practice of Pharmacy 1995, edited by E. W. Martin, Mack Publishing Company, 19th edition, Easton, Pa.

Compositions of the invention are useful in supporting joint health, bone health and strength, and athletic performance or treating joint deteriorations. As used herein the term “treating” or “treatment” of joint deterioration includes: (1) preventing joint deterioration; (2) reducing the rate and/or the amount of joint deterioration; (3) relieving the joint deterioration, i.e., causing regression of joint deterioration. As used herein the term “supporting” or “support” of joint health, bone health and strength and athletic performance includes: (1)beneficial effects on bone formation and quality; (2) improves mineralization process in bone; (3) contributes to healthy bone cortex and bone matrix; (4) aids in formation of collagen found in ligaments, tendons, cartilage, and bone; (5) decreases incidence of bone-releated injuries.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

While there are numerous commercial products that include glucosamine and chondroitin for supporting joint health, none of these products have shown to have any beneficial effects on bone health or strength. By only allowing improvement in joint health, a recipient of these commercial products could be at risk of injury due to poor bone health or strength. Silicon has been shown to increase bone health and strength, provide joint support, and improve athletic performance. In addition, silicon aids in collagen formation, an important part of bone and joint health. Moreover, silicon increases the glycosaminoglycan levels in cartilage, which is important in the prevention and treatment of degenerative joint disease. It has been shown that horses with increased plasma silicon concentrations had faster average race times, covered more distance before injury and completed more strides before injury. It has also been shown that supplemental silicon decreased the incidence of bone related injuries in young racing quarter horses.

Compositions and methods of the invention improve joint support, bone health and strength and improve athletic performance by combining therapeutic levels of glucosamine, chondroitin, and silicon.

In some embodiments of compositions of the invention for joint support, bone health and strength and improved athletic performance the daily dosage for humans is from about 2 mg to 15 mg of silicon, from about 250 mg to 2,000 mg of glucosamine, and from about 100 mg to 1,500 mg of chondroitin. It should be appreciated that the suitable dosage range typically depends on body weight and initial dosing versus maintenance. In veterinary use, e.g., suitable dosage for the horse can range from 15 mg to 150 mg of silicon, from 7,200 mg to 14,400 mg of glucosamine, and from 1,200 mg to 2,400 mg of chondroitin. For the dog, suitable dosage can range from 2 mg to 20 mg of silicon, from 2300 mg to 1800 mg of glucosamine, and from 125 mg to 700 mg of chondroitin. For the cat, suitable dosage can range from 1 mg to 10 mg of silicon, from 125 mg to 250 mg of glucosamine, and from 100 mg to 200 mg of chondroitin.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition for promoting joint health, mineral metabolism, bone health, bone strength and/or improved athletic performance, said composition comprising a therapeutically effective amount of silicon and glucosamine.

2. The composition of claim 1, wherein said composition comprises from about 0.1 mg to about 1 gram of silicon.

3. The composition of claim 1, wherein said composition comprises from about 10 mg to about 100 grams of glucosamine.

4. The composition of claim 1 further comprising chondroitin.

5. The composition of claim 4, wherein said composition comprises from about 5 mg to about 100 grams of chondroitin.

6. The composition of claim 1 further comprising methylsulfonylmethane

7. The composition of claim 6, wherein said composition comprises from about 5 mg to about 100 grams of methylsulfonylmethane.

8. The composition of claim 1, wherein the ratio of glucosamine to silicon ranges from about 600:1 to about 7200:1.

9. The composition of claim 1, wherein said composition is orally administrable.

10. The composition of claim 1, wherein silicon is selected from the group consisting of potassium silicate, sodium silicate, calcium silicate, sodium aluminum silicate, sodium zeolite A, orthosilicic acid, and a combination thereof.

11. A method for increasing joint health, mineral metabolism, bone health, bone strength or performance in a mammal, said method comprising administering to the mammal a therapeutically effective amount of a composition comprising silicon and glucosamine.

12. The method of claim 11, wherein the mammal is selected from the group consisting of human, non-human primate, equine, bovine, canine, feline, caprine, ovine and rodent.

13. The method of claim 11, wherein the composition further comprises chondroitin.

14. The method of claim 11, wherein the composition is administered to the mammal once a day.

15. The method of claim 11, wherein the composition is administered orally.

16. The method of claim 11, wherein the composition is administered parenterally.

17. The method of claim 11, wherein the composition comprises from about 0.1 mg to about 10 grams of silicon.

18. The method of claim 11, wherein the composition comprises from about 10 mg to about 100 grams of glucosamine.

19. The method of claim 11, wherein the ratio of glucosamine to silicon is from about 600:1 to about 7200:1.

20. A method for improving a joint performance, mineral metabolism, bone health, bone strength and athletic performance in a mammal comprising administering to the mammal a therapeutically effective amount of a composition comprising silicon and glucosamine.
